SEQUENCE LISTING (42 sequences)

SEQ ID NO: 1 - DNA, 142 bp, Artificial Sequence
  Probe
ggccgcagcctccactctcatgacaaccaagcccagccctgggccagggcctctgcgcgtgcgcgaccccgccccaccccaggccgcgcctgcgcacaaggggagggctccgctactctggccctcagggttgtgtagtttc

SEQ ID NO: 2 - DNA, 183 bp, Artificial Sequence
  Probe
ggaaattacctagcgaggagcggcttatccattgttgccaatgtttcaactgcgcggaaaccctgaagaaagtgcggagcacagggcacgcgggccgctcactgcccttccacgcgcccccacgtttctggccgctgcgctccttgccgtgcttttacaatgccacaggccactgggggagac

SEQ ID NO: 3 - DNA, 119 bp, Artificial Sequence
  Probe
ggcgaggaaatacatgcactggctgagaatcgcccgcgccagggcgcaacgccacaaggtgtagggagtgtgcggggtggggcgaaaggggacccaagagtccctgtggctcggagtgc

SEQ ID NO: 4 - DNA, 144 bp, Artificial Sequence
  Probe
ggaagttttgctgctagtcgcggacgcaatggcttcaaggttacttcgcggagctggaacgctggccgcgcaggccctgagggctcgcggccccagtggcgcggccgcgatgcgctccatggcatctggaggtactcgggtctc

SEQ ID NO: 5 - DNA, 587 bp, Artificial Sequence
  Probe
ggcgaggacgggccgtctgtgctgacccgcgaatagtgatcccagaggaagaacgcgtcctgagtattccaagtgcgagcagtgccacctgttctcggagatggcactgctcagcacgattgtccttgccagtcgtgctctggcagtgaagaggacttgtgaaatataattttcctctagaaggcactgcaccttcccatttaccgtcactttctcccgtttccacccctttccatcagtcacgttttcttcttttcgcagaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggtcagtaaatgttcatggaatcagatgaatgagtacatgacttcggaatcttaattatgtttctatgttcaaagatggaagggcttaatgtcttcctagagtttaaaagtgatgtttagtttttgcagaccccccaccccaaaataatatagtcttaaaaaaatttctggcc

SEQ ID NO: 6 - DNA, 100 bp, Artificial Sequence
  Probe
gcggaaataccccagcgcgtgggcggagcagcggcccgcagagggaggcggtggcgcccacggaacagccgcgtctaattggctgagcgcggagccgccc

SEQ ID NO: 7 - DNA, 132 bp, Artificial Sequence
  Probe
gggggcatccccctaaacttcggatccctcctcggaaatgggaccctctctgggccgcctcccagcggtggtggcgaggagcaaacgacaccaggtagcctgccgcggggcagagagtggacgcgggaaagc

SEQ ID NO: 8 - DNA, 206 bp, Artificial Sequence
  Probe
ggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcaggtaggtggcaggacc

SEQ ID NO: 9 - DNA, 100 bp, Artificial Sequence
  Probe
gttcttctcgattaccgagtgcctctcctgaaagcaagtcagcgtcgcctaacctcttcagcttcgaaatggcggccaccagatcgctaggccacgcccc

SEQ ID NO: 10 - DNA, 1684 bp, Artificial Sequence
  Probe
ggggtgactggctctagaaggaaaagacataaaacagataaccaactgtttttatgaaatgtattgtccaaggtcaattcaaaaaaattatttaacaaaattacttattttaccaaactcatgtatcttgataatgtatttttctttgatttaatgatagtgtttgtcttcatttcacagaagttattcatctcttatttttggtgagacaactagaacaatttctgctttatgaaacatgcaaataaaaattaggagcaatgttaaatttaactaacttggtttaatgctagtcaaccagtaattcttcagtacacttaatatgatgggaaaaacagcatgctgcattgttgcaaattttacattttctttaataaaatgtatttgtttacggtgtcagtagggattaaaaatcataattctaaatttaaagtatttaaattatagaaaaatgattccattattttggtaatggaacagagaaatacaaatatagaaacgtttttatcctttagatcttctaatggtgcagatatttcttaaccaatttcaagagtgccttattcaaaaacaaaaaacctgcacatagaaaaagcaaacatgtttttttaaaaaaagccctgtaaaagtggttaatctgtcgtctgattttcaatgtatgactttaatcaattgcatattccttaattctgcaaatgacaattaaaattaggaatcagtacctgaaaacgccatttatacttttgagtttatattagaaacacggttttgatgaaatacctttttcggttcaatctttttgaattgtgaccacaaccaatagctacacttagattctacagaattagtaggaaagatacaaatgagaaggtctttctagacaatggatctcactgaacatcatcttagtgtgtagactttttccactgcagtggcccacatgtgctatggtgacagacggcagttggcattaccacttatatagtaaaatatctttaagacaaaaataaaagttatttaaagaatacgctggccctgaaacatgagctgtgccttaaactacatatcctacctccatgagtgaatgaataagtgggttttccgcctgcatctatgtgttaagacctattgtttgcttgtattaattgtagtcctgggataaaagcacagaggtaactttcactgctgctttttgtactctctccaatgttttggaggaaaaataagcacaaacaatagcctagagaaactgaatcgatcatacttgttgcagcttcgacaaacgtcaattttgctgcattagaatgggaaacatttttcagtctattgaaatgaattacaaacgtttttaaatagaaaattagttaaaaaattggaggctgcttaatcgatagctttctctataaacatacttggatttcacaaataagtaataccgtaaaaatcttcttctccaaagaaaaatccccacaaataaatctattgatacctagtgacaagtggaaccagataaaaatggaatctataagaattaacctaattgacagcgctctggagctaatccatttccattagttatttgttcacagtaggtactcctaaggacttgttgaattgcgggcttggcgcccgttctacggagagttcacagccttcgtgagtggggacagaaggcggctcggcc

SEQ ID NO: 11 - DNA, 130 bp, Artificial Sequence
  Probe
gagttcccgtgatgccccacgcggctgcaatgattggttaagggttttgcttttagggcgtggacgggctcctgagcaatagtggatgagctgtgagtgcgcgcgcgtgcgcggggccgcgacctgtgcc

SEQ ID NO: 12 - DNA, 135 bp, Artificial Sequence
  Probe
ggctcgcgggaggctgtcggggtaatggccacacgctgacagaaccagccgagtggaaaaggggagcgaagccgttcctctgcacccttccccaggcctgaggccttcccgcttggtgctgccgccgccactgcc

SEQ ID NO: 13 - DNA, 123 bp, Artificial Sequence
  Probe
gggaaatcgacctctatcggtagccatagcttccccttcgctaagaactgctgtaagcacgaaatcgacatcaatagaaacccttccgcgttcctcggtaactaacggcaaccctgcgctccc

SEQ ID NO: 14 - DNA, 378 bp, Artificial Sequence
  Probe
gggccgaaggccaatcacactgcagctaggtcttgcgattggacggcagtgagagccgattggccgccgccgcgagtttcgggctccctccctctcccttttgccccaactccagcgcctaagcttcaggccaatgagacagcgctttatagacgccctcccttcgctttcttctccccaccttggagagggaggggaagtcctgactggccagactgtcctcggaagcccccttctcttcaccaatcaccgaaggaggtacgctcccagcggagttttccagccaatcacaaagcggcggcggcgcccagccgtgcagtttcaccagcgtctctgggtttcaccgtcctcaactcttcaagcctcttcgtaagggcc

SEQ ID NO: 15 - DNA, 128 bp, Artificial Sequence
  Probe
ggccgcgcacaccaagcgcagcaagcctctgtatcaatggcccccgcggctgggcggggcgcggccgcccaaaggcgctgcctttctggaagctttgttcccattttagcgtctgagagcttgcagcc

SEQ ID NO: 16 - DNA, 636 bp, Artificial Sequence
  Probe
ggttctcaacttaacagcatgtggcccagcacccgcttttgccttttattccgcactctgatttttggggggatcgtttgttcccgctcattttctacaaacattacggtatcctgttagcattccgaacaaggggctgttcatatatttgccttcaatgatttcctgaagggacatgtggaagtaatagcgagcggtgcagtcacccaagctgaagatgcacgagagcgggtcgcgtagaagaactgcggcaatgggacccccagcgcagccccaggcgccctgttttcgaggctgacatcccaagatcctgtcctggttaggcacagcaccgcgcccgttggagtacttctctgtgactctcccctgtccactgcactgtgtccaccctcctggtgaaggaggagacggctgttggacatgccagtcccaacctggccactttggccattttcagagtgcgtgtgttggggtggggttgagggagaaggatgaaggatgacccccaaccccaacaggagaattcaaaaatcagagagcgatgaaggtgaacgcgctcccctcccccgctgtcgctgggaagccccactctgcccaggtctggttcctggagcatcagcgccccctttccctcgc

SEQ ID NO: 17 - DNA, 149 bp, Artificial Sequence
  Probe
gaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatccc

SEQ ID NO: 18 - DNA, 491 bp, Artificial Sequence
  Probe
gccccccgcgccctttccgaggttccctgccttgacttccccgagttctacgacaacctcagcgacagcgggaggagctgggcaatcggttttgcagggcaaactccaggctctcctcatttattattctctgggcggcgcacgggagaccctgcagggcaacagagcagagcgactacagctcccaggagccaacgctgcagggctgagccgacgcgggggacagacaggacctaaatggtggtcggaggaagggcaaggcagacctcttctgttaccttagcgccagcaaaccgagccgccgccgtcgccaagccaggagaccaacccccagtctcccgactaagtatttttaaatctggcggggctttccttcccgagccaaccactcccaccgtttacagccccaaccagtcagcgtgaggcccgccatttttcaaaccctcttcccgccgccaatcaggatcgagcagtacattcctctcctgacc

SEQ ID NO: 19 - DNA, 121 bp, Artificial Sequence
  Probe
gggccgcggagcgtgtgacgctgcggccgccgcggacctggggattaatgggaaaagttttggcaggagcgggagaattctgcggagcctgcgggacggcggcggtggcgccgtaggcagc

SEQ ID NO: 20 - DNA, 323 bp, Artificial Sequence
  Probe
ggaccacggctattgccagcccatctccatcccgctgtgcacggacatcgcgtacaaccagaccatcatgcccaacctgctgggccacacgaaccaggaggacgcgggcctggaggtgcaccagttctaccctctagtgaaagtgcagtgttccgctgagctcaagttcttcctgtgctccatgtacgcgcccgtgtgcaccgtgctagagcaggcgctgccgccctgccgctccctgtgcgagcgcgcgcgccagggctgcgaggcgctcatgaacaagttcggcttccagtggccagacacgctcaagtgtgagaagttcc

SEQ ID NO: 21 - DNA, 253 bp, Artificial Sequence
  Probe
ggcgcgtcggagcgaggcaagttctcctgcccgcgcgccctcaaggtgccctcctacctcaactaccacttcctgggggagaaggactgcggcgcaccttgtgagccgaccaaggtgtatgggctcatgtacttcgggcccgaggagctgcgcttctcgcgcacctggattggcatttggtcagtgctgtgctgcgcctccacgctcttcacggtgcttacgtacctggtggacatgcggcgcttcagctacc

SEQ ID NO: 22 - DNA, 159 bp, Artificial Sequence
  Probe
ggccatcaagaccatcaccatcctggcgctgggccaggtggacggcgatgtgctgagcggagtgtgcttcgtggggcttaacaacgtggacgcgctgcgtggcttcgtgctggcgcccctcttcgtgtacctgtttatcggcacgtcctttctgctggc

SEQ ID NO: 23 - DNA, 186 bp, Artificial Sequence
  Probe
ggaggcggagcatccgcgcggggcctggcgcgcctgcgccctgcgcccgcccctcgccgtaggaggaggtggaggaggaggcggctcgggagagcgagcagcgagctggctggatcgccgagcgcgagtgagggagccgagccgcccgccgccgccgcctccgcctcccctccgcgaacaggagcc

SEQ ID NO: 24 - DNA, 290 bp, Artificial Sequence
  Probe
ggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttc

SEQ ID NO: 25 - DNA, 101 bp, Artificial Sequence
  Probe
ggctgggtttctgggcagaggccgaggcttagctcgttatcctcgcctcgcgttgctgcaaaagccgcagcaagtgcagctgcaggctggcggctgggaac

SEQ ID NO: 26 - DNA, 196 bp, Artificial Sequence
  Probe
gggtgtcatccgcggcgctggactgtttcattttgagtttgcaacttgggtttttcagcgagctttttttcttcctgaaagctaatggcttccacagcaattagacattttcctcgcccgccccttccctcccctttctttacatataggagatgggatactcattcccgctgctattgataaggtcggaggcggc

SEQ ID NO: 27 - DNA, 160 bp, Artificial Sequence
  Probe
gtgtaaacagcgttcaaaaattccgccctcccaaaaaaaggatgctgaaaccgatttccaaccccgaagacggagctcatcaccccgcaacgggatccgccgtggctccccgcgcgtgcaccgcggctgggcagtctcaggtggctgggggcgcgagacc

SEQ ID NO: 28 - DNA, 192 bp, Artificial Sequence
  Probe
gggtggcacagtttttatggttagggtgcggatccccttcctgagcctgagctatcatacgtcccaccaggggtattgtgaacgtagcctcctccacgaacctgcttacagactccaagagtctgcaactggtactcgagcccagtctgcagctgttgagccgcaaacagcggcgtctgatacgccaaaatc

SEQ ID NO: 29 - DNA, 282 bp, Artificial Sequence
  Probe
ggcccgctgggtgcctcgactccccgcgttccccgctgctgcgaaggccgtggccctcgcctgcacaccgcgcccaggctcggtggctcttaactccgcgccccatgcacgccccctctctccctccttgactcctcccagcaccccccttctcctacccgctccatctggctttctgccccccatgccccgcctccccgtggccaggtgtcctgggtccccaggagcccctcgcccgagggacagagacagccccaggcaagttgaaggtccgagagcccc

SEQ ID NO: 30 - DNA, 255 bp, Artificial Sequence
  Probe
gggctcacgcgcgggttctcgaaaatcagctgtttcagtcttgggctagtccactaattggactcctcccctcgtagaaagtgcctacttgaacttctccaccaatcgctgaagctgcaggtgtggtttcggctcagcttgtcccgccctgcggaggggcggagttgcggcggcgccagtgagctcgcagtctgggaagggcttgactgaatggcagccagtgtcggggtggcggctgggaatgggggccgctcc

SEQ ID NO: 31 - DNA, 1162 bp, Artificial Sequence
  Probe
gcaccgaccgcacagcagcgaccccccgtctgggacggcttcacctctggacccccgacctcggggacccatcaccatctcccagaagggccccagggaccagcccgcccctcagggacccaccccgtccgctgcagggacgagacttcctgcggcagaccacctcgtcagctacgggaccctcacctcgcagacctctccccacccccacctcacagatcgaccgcccaactcggggagcagctgcccgatctcatgggctgctccctcatttcctcgacctgctccccgtccctccagggccgccccccacttcacagactagcaccccccttcagggataacccctcggagcccacccctttcatttcacaagtcagatgcaaaagctagagatgagcgtgtgatgaccacacccccatttcaaagacaagtttcccagtctgcctgctcttctgaccccccacttcaaatctggtcaccccgtttcaaggacccatcactcattttctctcactgacctccccgctctgtggaccagcccctcaattcacagaccagtccgtctaccatggtatctaagtctagaacggtaatttgggagtcctgattctctccccacccctagattctggtgaaatgccaggggaatgaggtggaaggtgggccccaaggtatgaccatccctgaacgtgtcctggctagcagagtcccctccaccctctctgcgtcaggccctgctggaagccacgcaggtgaaagaggacaggatgcccagaacagagttttactgttttcctgttattggagccccaaggctactcttttcagtccagtcttgatctggtgttaggggaaagaaagtatctcctggaagccgactctaaacccaagggggttggtgccagggttctaggaatgccttgggtggcgtgaacgcaggagtgggaggattcgctgtctctccctccccttcacgcctgtagctggagggccctgagcttgggaggtgaggaggggggcaacgtttcttctctgcaggagccttgtctttcctctctggactgtgggtggcttgtgtgaccactgatgcttcctctgtcccaggctctgaggagtagattcagcagacaggactgatggacctctaccaagcagcgcc

SEQ ID NO: 32 - DNA, 588 bp, Artificial Sequence
  Probe
ggtgcagatacggggttgctcttttgctcataagaggggcttcgctggcagtctgaacggcaagcttgagtcaggacccttaattaagatcctcaattggctggagggcagatctcgcgagtagggtacaaggcactatgaaatgatctagtttcgtgggtgaggggctgaagggcctatgatgcacggaggcggggaaaggatttagagataacgtggtttgaaaggcgggacctggtgcggggacgctcttgggaggagtcttctccccagccttagctggtttcatgatttctttgcgtctgtaggcaacgcggtaaaaatattgcttcggtgggtgacgcggtacagctgcccaagggcgttcgtaacgggaatgccgaagcgtgggaaaaagggagcggtggcggaagacggggatgagctcaggacaggtaagggaatgaaatcagcccttcttcctagaagctgcggcgggggtgtttgtcattcccttgatgtacggtaagtacgggccgactcatttttgcaggggtttgtgaagaagtcgcaggaaccgtaggctttcgttgggtctatagttaacgc

SEQ ID NO: 33 - DNA, 251 bp, Artificial Sequence
  Probe
ggagcggcgcctgcgtcagcgcagacgtcagggatatttataacaaaccccctttcaagcaagtgatgctgaagggataacgggaacgcagcggcaggatggaagagacaggcactgcgctgcggaatgcctgggaggaaaagggggagacctttcatccaggatgagggacatttaagatgaaatgtccgtggcaggatcgtttctcttcactgctgcatgcggcactgggaactcgccccacctgtgtc

SEQ ID NO: 34 - DNA, 766 bp, Artificial Sequence
  Probe
ggcaccattggtacacggaccacattttgtttcccaaagcaaccagatgatttttgaggtcctttgcagctcgaacacggaatgactttgtggtgatcgagtagccagaagttcacggaatgcactgtcacaattgtgattccgccatagcgccgtgcatccatccaacacttgtttaatacctatatttaatacctaataccttagacggtcctaggctgtggacacagaagactaaaccccacttcctgagttgaagtgggggaaatagaggagtaaatcatttcacgatgtgtggttaagtgctgcagctcaggtaaccaccagtacacagaggaagggcggtttctggagacagggcggagaggagaccgtgggtgggcatttcccagagcagcctctgccagccaccctactttgatcactttgccgccgagcagcagcggcggtcaacctcagcctcccttagcaacctgagtgccccgtccaggagccttactattggtctcatgcagcgagatgggcagttctgccgtgcaatcccagctcgcagttcttgctccgcgtgtactcacgggaggactcgcaggcgttaccgccttcccgcgtgccccgcccaccctcccgagcgtctttcggcgcatgcgcggactggagctgtgtgcagggccagcgcggagcccgagcagccgcggtgaagcgcctgtgctctgccgagactgccgtgcccattgctcgcctcggtcgccgccgctttagccgcctc

SEQ ID NO: 35 - DNA, 278 bp, Artificial Sequence
  Probe
ggcagggaggaggcgcttgtggggggcacccacggggcacagtgatccctgggggtctgcggacctcctgggccccgcagcagacacgagtttagcctttgggtttagtttaaatcacataagggtgtcgtgcaatcgatttatggtttctacacaccagacactttaacctccaaccccccccatccaaagccaacaagaaaatgcggtgccgtgttggcagctgagctgcgcccgaagagacgcagggagacgtaagagaggaaagtgtgagtggc

SEQ ID NO: 36 - DNA, 166 bp, Artificial Sequence
  Probe
ggcagcgcggggcggccgcttctcctgtgcctccgcccgccgctccactccccgccgccgccgcgcggatgccaagcaccagctttccagtcccttccaagtttccacttggccctgcggctgcggtcttcgggagaggagaaactttggggcccgcgccgcgcgc

SEQ ID NO: 37 - DNA, 238 bp, Artificial Sequence
  Probe
ggcgatttggggacgggcgaggctgggccgtcaggaccacagagggcacgtgggagggaggaagggaggcttccttgggagtgaaggatgccccgtgtcttcctgagcgaggcagagccaccctctggcgccaagatcgcggtttgggggttctgcgccccccacccccaccccgagctcgtggcggcgtctccctcgcgccgcgcccctgaattccccagggccgagcccgtcacgc

SEQ ID NO: 38 - DNA, 370 bp, Artificial Sequence
  Probe′
gggtccacagggatgcgaaggagcgggacaccatgaaggaggacggcggcgcggagttctcggctcgctccaggaagaggaaggcaaacgtgaccgttgtgagtacaaaagagacaggttggggagcatcccccccatctcacctgggtacccgacttggctctgcctacgggggcgggggtcccttgggcaggaacggagctcataacctgatcagctttctcttcttctctctgtttttgtcttgtttggtgtgtttccttggggtcatgggggtggcttcatgttagtttttgcaggatccagatgaagaaatggccaaaatcgacaggacggcgagggaccagtgtgggagccaggtaggtccgcc

SEQ ID NO: 39 - DNA, 462 bp, Artificial Sequence
  Probe
ggtgcctgtgttgttgggcaaagccaggggaaaggtcaggtcgccttgggatctggcctagcactgagtgcagaggaagagaagcttctgagatcgagaggcagcggctacaggaggaggaagaaagcaaggcggggaaaaccaaagttcaggagagcatttgcaggagggcgctgcggggtagggtctgagtcggagccccgaatcggactcgagtcctgtcgcgtggggacggagcgtgcagaggcccatgcgagggacagacacaaaggccctgggagctgcaggtcagaccactggacagcgcggccgcggacctaacgcccctgtaaggggtgctcacgcttggggggaactctccgaaagagaggaccactgaaagccaccctggcagcaggggcggaacagacagacgtgggtctggccgtcgccagaactgcctgcggaccagccccgcgctgc

SEQ ID NO: 40 - DNA, 132 bp, Artificial Sequence
  Probe
gggaacggcgcgcgctcccgccgccatcgcgcctccgtccccgcctgcggcctgtctgggggtcgcggggcgcaggcgcggcgggccgacgggcgggggtcctccccacgggtgcgcgggcgcctttgttcc

SEQ ID NO: 41 - DNA, 121 bp, Artificial Sequence
  Probe
ggtagtgctgggcgtggcctcgggactacatatcccagtggcccccgtgcggcgactttagctgctgctgtctcagccgctccacagcgacggcagcggctgcggcttagtcggtggcggc

SEQ ID NO: 42 - DNA, 805 bp, Artificial Sequence
  Probe
ggcgcagcgcggcctttaaagtctggaccctcgggaaggcaacggaagggtccccacccctcaatttgctagtatcatggccttcttttggactttcgattgtaaaactgtgaattccatcagatgcttgtcggtgttggcggcggtggctgctgaggtcctgaaagcgttttgcctccgcgtgtgtgcagaacaccgaaataatcgtttttctctcttcgcctctgtttgcagccaacggctttcattaaatatcctcagcggtgctaatggtggtctccgcgcaggctttctaaaatggattactgacgccagtgttggtggcatatttccgtcttgaagtttttacttctaagtggattttaatatcgtactgctggcgtttcaggaaggataaactaacattactctcttgatttgttaaagatccgtttagagttgggcctaatttgactttttttttagcccccaagtgagctcttttaccagcataaatgctcccagatgttttagcccaattgctatggactcctgttaatgatttagtggtcattatgtatgcagccttaatggagttcttgcagaatcacacgtagactccctcatcttgattcaggtcccttagaaaagtgaaatgatagtgacggttttttttttccccccgagacggggtctcgctctgtctccaggctgaagtgcagtggcgtgacctcggttcacttcaacctccgcctcctgggttcaagcgattcttcttcctcagcttcccgagtagctgggactacaggcgcgcgacaccacaccc

(19) United States
(12) Patent Application Publication
(10) Pub. No.: US 2013/0090257 A1
(43) Pub. Date: Apr. 11, 2013

(54) PATHWAY ANALYSIS FOR PROVIDING PREDICTIVE INFORMATION

(71) Applicants: Nilanjana Banerjee, Armonk, NY (US); Nevenka Dimitrova, Pelham Manor, NY (US); Robert Lucito, East Meadow, NY (US)

(72) Inventors: Nilanjana Banerjee, Armonk, NY (US); Nevenka Dimitrova, Pelham Manor, NY (US); Robert Lucito, East Meadow, NY (US)

(73) Assignee: KONINKLIJKE PHILIPS ELECTRONICS N.V., Eindhoven (NL)

(21) Appl. No.: 13/704,070

(22) PCT Filed: Jun. 21, 2011
(86) PCT No.: PCT/IB2011/052705

§ 371 Date: Dec. 13, 2012

Related U.S. Application Data
(60) Provisional application No. 61/359,399, filed on Jun. 29, 2010.

(51) Int. Cl. G06F 19/18; C12Q 1/68

(52) U.S. Cl. CPC G06F 19/18; C12Q 1/6809
USPC 506/9; 435/6.11; 702/19

(57) ABSTRACT

A method for assigning ranking scores to pathways in a set of pathways for classifying patients is disclosed. The method comprises the steps of comparing biomolecular datasets from different groups of patients and performing an analysis in order to assign ranking scores to pathways in a set of pathways. Furthermore, a method for using cancer pathway evaluation to support clinical decision making is disclosed. This assessment is further used for stratifying ovarian cancer patients based on chemosensitivity to platinum based drugs, the standard chemotherapy. We present the method for evaluation and ranking of the most relevant pathways responsible for platinum sensitivity. Clinical decision support software system should be able to then visualize this information for a clinician, contextualize it within a patient data set and help make a final decision on the potential responsiveness.

### FIELD OF THE INVENTION

The present invention relates to a method for pathway analysis, and more particularly to a method, an assay, a clinical decision support system and a computer program product for pathway analysis for providing predictive information in relation to cancer.

### BACKGROUND OF THE INVENTION

Ovarian cancer is the most lethal of all gynaecological cancers due to its late diagnosis, high mortality and low 5-year survival rates. Reasons for this poor outcome include non specific presenting symptoms and identification in advanced stages of disease, mainly due to there being no reliable screening methods for early detection. Ovarian cancer is the 6th most common cancer world-wide with 204,000 cases and 125,000 deaths worldwide. The exact cause of developing ovarian cancer is still unknown; however, women with certain risk factors may be more likely than others to develop ovarian cancer. The top ranking factors include age, parity (like for breast cancer), personal and drug history. For the approximately 10% of familial linked ovarian cancer, mutations in BRCA1 and BRCA2 appear to be responsible for disease in 45% of families with multiple cases of breast cancer only, and in up to 90% of families with both breast and ovarian cancer. An Open Access On-Line Breast Cancer Mutation Data Base serves as a repository for over 2,000 distinct mutations and sequence variations in BRCA1 and BRCA2.

There is evidence in the medical literature about the role of DNA Methylation in cancer.

The highest sensitivity for hypermethylation is detected in the following genes: CDKN2A, PCSK6, OPCML, SFN, CTCF, ESR1, DLEC1, RASSF1A, GATA4, RUNX3, WT1, MYOD1, and PYCARD. Although less frequent, there are also genes that are hypomethylated and overexpressed in cancer samples, and are potential oncogenes. Synucleins are a family of small cytoplasmic proteins that are expressed predominantly in neurons and retina. In a group of SNCG mRNA-expressing tumours, there were 75.7% (25 of 33) cases with hypomethylated or demethylated exon 1 of SNCG. The genes include synuclein-gamma (SNCG). Hypomethylation of the RHOA promoter region in tumour DNA was observed two times more frequently than increased methylation.

Regarding predicting treatment response, information about how a cancer develops through molecular events could allow a clinician to predict more accurately how such a cancer is likely to respond to specific therapeutic treatments. In this way, a regimen based on knowledge of the tumour's sensitivity can be rationally designed. Thus, characterization of a cancer patient in terms of predicting treatment outcome enables the physician to make an informed decision as to a therapeutic regimen with appropriate risk and benefit trade-offs to the patient.

In terms of diagnosis, the key to improving the clinical outcome in patients with cancer is diagnosis at its earliest stage, while it is still localized and readily treatable. The characteristics noted above provide means for a more accurate screening and surveillance program by identifying higher-risk patients on a molecular basis. It could also provide justification for more definitive follow up of patients who have molecular but not yet all the pathological or clinical features associated with malignancy.

US20090011049 is related to the area of cancer prognosis and therapeutics. In particular, it relates to aberrant methylation patterns of particular genes in cancers. For example, the silencing of nucleic acids encoding a DNA repair or DNA damage response enzyme can be used prognostically and for selecting treatments that are well tailored for an individual patient. Combinations of these markers can also be used to provide prognostic information.

While there are many genes reported to be differentially hypermethylated in ovarian cancer, currently there is still a need for methods which are able to predict a course of events for patients suffering from or being examined for ovarian cancer. For example, there are no diagnostic methods which are able to predict therapy response to platinum based drugs. The primary chemotherapy agents used in the treatment of ovarian cancer are cisplatin and carboplatin. The mechanism of platinum sensitivity is still not well understood in the literature. We need clinical tools that will assess early resistance to platinum so that the patients can be given alternative therapy choices with higher chance of better outcome.

Hence, an improved method for providing prognostic information would be advantageous, and in particular a method for providing prognostic information earlier, more efficiently and/or more reliably would be advantageous.

### SUMMARY OF THE INVENTION

In particular, it may be seen as an object of the present invention to provide a method that solves the above mentioned problems of the prior art with the inability to provide predictive information at an early stage, such as being able to predict therapy response to platinum based drugs at an early stage.

It is a further object of the present invention to provide an alternative to the prior art.

Thus, the above described object and several other objects are intended to be obtained in a first aspect of the invention by providing a method for assigning ranking scores to pathways in a set of pathways for classifying subjects, said method comprising the steps of
obtaining a plurality of primary datasets comprising biomolecular features from a plurality of primary subjects,
obtaining a plurality of secondary datasets comprising biomolecular features from a plurality of secondary subjects,
identifying a clinical parameter, where the clinical parameter is a parameter relevant to cancer and which has different values for the primary subjects and the secondary subjects,
identifying a plurality of stratifying features in the primary and secondary datasets which stratify the primary and secondary subjects,
identifying a plurality of stratifying genes corresponding to the stratifying features,
assigning a ranking score to each pathway in the set of pathways thereby providing a set of ranked pathways, said ranking being based upon the plurality of stratifying genes.

The invention is particularly, but not exclusively, advantageous for enabling a physician to classify a sample, such as sensitive and resistant samples, such as normal and tumour samples, based on datasets comprising biomolecular data. The invention provides a tool for biological understanding. This approach relies not only on individual genes, but involves pathway analysis. It is important to have the tools for biological understanding such as pathway analysis to be applied in, for example chemosensitivity, when making therapy plans for cancer patients.

The various steps of the invention may in certain instances be interchanged or combined as is understandable from the principles of the invention.

In an advantageous embodiment, the invention may be utilized for visualization of stratifying genes within a plurality of pathways. In a particularly advantageous embodiment, the visualization may further be based on biomolecular data being obtained from a patient or a sample.

As used herein the term “expression” shall be taken to mean the transcription and translation of a gene. “Expression” or lack thereof is often also a consequence of epigenetic modifications of the genomic DNA associated with the marker gene and/or regulatory or promoter regions thereof. Genetic modifications include SNPs, point mutations, deletions, insertions, repeat length, rearrangements, copy number variations and other polymorphisms. The analysis of either the expression levels of protein, or mRNA expression are summarized as the analysis of ‘expression’ of the gene. Also, the analysis of the patient's individual genetic or epigenetic modification of the marker gene can have impact on “expression”.

In the context of the present invention the term “chemotherapy” is taken to mean the use of pharmaceutical or chemical substances to treat cancer.

In the context of the present invention the term “regulatory region” of a gene is taken to mean nucleotide sequences which affect the expression of a gene. Said regulatory regions may be located within, proximal or distal to said gene. Said regulatory regions include but are not limited to constitutive promoters, tissue-specific promoters, developmental-specific promoters, inducible promoters, as well as noncoding RNAs (such as microRNAs) and the like. Promoter regulatory elements may also include certain enhancer sequence elements that control transcriptional or translational efficiency of the gene. These sequences can have various levels of binding specificity and can bind to so called transcription factors as well as DNA methyl-binding proteins, such as MeCP, Kaiso, MBD1-MBD4.

In the context of the present invention, the term “methylation” refers to the presence or absence of 5-methylcytosine (“5-mCyt”) at one or a plurality of CpG dinucleotides within a DNA sequence.

In the context of the present invention the term “methylation state” is taken to mean the degree of methylation present in a nucleic acid of interest, this may be expressed in absolute or relative terms i.e. as a percentage or other numerical value or by comparison to another tissue and therein described as hypermethylated, hypomethylated or as having significantly similar or identical methylation status.

In the context of the present invention, the term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

In the context of the present invention, the term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

In the context of the present invention, the term “methylation assay” refers to any assay for determining the methylation state of one or more CpG dinucleotide sequences within a sequence of DNA.

In the context of the present invention, the term “pathway” refers to the set of interactions occurring between a group of genes, which genes depend on each other's individual functions in order to make the aggregate function of the network available to the cell.

In the context of the present invention, the term “biomolecular features” refers to a set of features which are of biomolecular character, such as a set of levels of gene expression or a set of DNA methylation levels.

In the context of the present invention, the term “primary subjects” refers to a group of subjects, such as mammals, such as humans, such as patients, such as samples, which are distinguished form a corresponding group of “secondary subjects” in that they can be associated with one or a combination of clinical parameters which differ between the primary and secondary subjects.

In the context of the present invention, the term “secondary subjects” refers to a group of subjects, such as mammals, such as humans, such as patients, such as samples, which are distinguished form a corresponding group of “primary subjects” in that they can be associated with one or a combination of clinical parameters which differ between the primary and secondary subjects.

In the context of the present invention, the term “primary datasets” refers to datasets derived from primary subjects, which datasets comprise biomolecular features.

In the context of the present invention, the term “secondary datasets” refers to datasets derived from secondary subjects, which datasets comprise biomolecular features.

In the context of the present invention, the term “clinical parameter” refers to one of a set of measurable factors, such as grade, hormone receptor status, that characterizes a patient and can contribute to the presentation of the disease. The clinical parameter may be any one or a combination of p53 status, ER status, grade, stage and a sensitivity towards the therapy comprising one or more platinum based drugs, such as platinum free interval.

In the context of the present invention, the term “stratifying features” refers to biomolecular features which differ in a statistically significant manner between the primary and secondary datasets.

In the context of the present invention, the term “stratifying genes” refers to genes which comprise stratifying features, i.e., genes which separate primary and secondary subjects.

In the context of the present invention, the term “ranking score” refers to a score representing a numerical value.

In the context of the present invention, the term “node” refers to a gene in a pathway.

In the context of the present invention, the term “connection” refers to the informational interactions between nodes in a pathway.

In the context of the present invention, the term “hub” represents a node with a number of connections being larger than an average number of connections per node in a given pathway.

In the context of the present invention, the term “important hub” represents a hub with a number of connections being larger than an average number of connections per node in a given pathway.

In the context of the present invention, the term “functional node” refers to a node in a pathway which is also a stratifying gene.

In the context of the present invention, the term “significance value” refers to the use of p-value where lower p-value, corresponds to a less likely chance that the null hypothesis is true, and consequently the result is more “significant” in the sense of statistical significance.

In the context of the present invention, the term “subject classification score” refers to a numerical value based on the difference between database values of stratifying features and values of corresponding features in the subject dataset. The subject classification score corresponds to a quantitative classification of the subject.

According to a second aspect of the invention, the invention further relates to an assay for analysing target nucleic acids comprising one or a combination of the genes taken from a group consisting of stratifying genes according to the first aspect, and their regulatory regions by contacting at least one of said target nucleic acids in a biological sample obtained from a subject.

This aspect of the invention is particularly, but not exclusively, advantageous in that the assay according to the present invention may be implemented by immobilizing gene sequences complimentary to said taken from the group consisting of stratifying genes according to the first aspect, and their regulatory regions onto glass-slides or other solid support followed by hybridization of labelled, such as fluorescently labelled, such as radioactively labelled, or otherwise labelled nucleic acids derived from the biological sample obtained form a subject (comprising the sequences to be interrogated) to the known genes immobilized on the glass-slide. After hybridization, arrays are scanned, such as using a fluorescent microarray scanner. Analyzing the relative intensity, such as fluorescent intensity, of different genes provides a measure of the differences in gene expression.

In an alternative embodiment of the invention nucleic acid methylation detection is performed using methylation specific PCR or methylation specific sequencing to assess the level of DNA methylation

According to a third aspect of the invention, the invention further relates to a method for classifying a subject, said method comprising
obtaining a subject dataset comprising biomolecular data, such as gene expression data or DNA methylation pattern data, of a target nucleic acid comprising one or a combination of the genes taken from a group consisting of stratifying genes according to claim 1 and their regulatory regions,
identifying the pathway according to claim 1, which is assigned the highest ranking score,
accessing a database comprising database values of the stratifying features corresponding to hubs according to claim 1,
calculating a subject classification score based on the difference between database values of the stratifying features corresponding to hubs and values of corresponding features in the subject dataset.

This aspect of the invention is particularly, but not exclusively, advantageous in that the method according to the present invention may be implemented by means of a processor adapted to carry out the method.

According to a fourth aspect of the invention, the invention further relates to a clinical decision support system comprising
an input for providing a subject dataset comprising biomolecular data, such as gene expression or methylation pattern, of a target nucleic acid comprising one or a combination of the genes taken from a group consisting of stratifying genes according to claim 1 and their regulatory regions,
a computer program product for enabling a processor to carry out the method of claim 15,
an output for outputting the subject classification score.
   This aspect of the invention is particularly, but not exclusively, advantageous in that the clinical decision support system according to the present invention may be implemented by software shown on a workstation, or a handheld computer, or phone, that shows the values for, for example differentially methylated pathways, potentially along with other clinical parameters obtained from the patient. In one specific example, significantly deregulated pathways may be shown. A Clinical decision support system according to an embodiment of the invention may be utilized in order to evaluate candidate pathways for carboplatinum based therapy in adjuvant setting for ovarian cancer patients. If a patient is found to be resistant due to de-regulated PI3K pathway, PI3K inihibitors could be administered to offset the deregulation and help the patient become more responsive to chemotherapy. In a particular embodiment, the output may further include the activity levels and deregulation with respect to normals, resistant to therapy and sensitive to chemotherapy of different pathways.

According to a fifth aspect of the invention, the invention further relates to a computer program product for enabling a processor to carry out the method according to the third aspect.

The first, second, third, fourth and fifth aspect of the present invention may each be combined with any of the other aspects. These and other aspects of the invention will be apparent from and elucidated with reference to the embodiments described hereinafter.

### BRIEF DESCRIPTION OF THE FIGURES

The method, assay, clinical support system and computer program product according to the invention will now be described in more detail with regard to the accompanying figures. The figures show one way of implementing the present invention and is not to be construed as being limiting to other possible embodiments falling within the scope of the attached claim set.

FIG. 1 shows clinical data: Platinum Free Interval (PFI) for all samples,

FIG. 2 shows hierarchical clustering of all loci after a t-test (p-value 0.05) and fold change >1.1,

FIG. 3 shows the Wnt Pathway and functional nodes that are important in determining response to chemosensitivity,

FIG. 4 shows -PI3K-akt pathway and functional nodes that are important in determining response to chemosensitivity,

FIG. 5 shows the PDGF signalling pathway and functional nodes that are deemed significant in tumor vs. normal analysis,

FIG. 6 is a flow-chart of a method according to an embodiment of the invention.

### DETAILED DESCRIPTION OF AN EMBODIMENT

In a further embodiment the invention relates to a method, wherein assigning ranking scores to pathways in a set of pathways further includes the step of assigning a ranking score to each pathway in the set of pathways comprises calculating a significance value for each pathway, said significance value being based upon a number of common genes between the plurality of stratifying genes and the pathway.

The significance value may be a p-value based on the Hypergeometric distribution or Fisher's exact test.

In one particular embodiment, the step of the step of assigning a ranking score to each pathway in the set of pathways comprises calculating a value based on gene overlap with stratifying genes. In an exemplary embodiment, the calculation of a significance value may be performed according to the following example. Suppose you have N genes, where N would be the number of genes in a chip, such as a chip used for generating primary and secondary datasets. M genes are annotated to a specific pathway in the set of pathways. n genes are found to be in the input list, such as comprised within the stratifying genes, for example differentially methylated. k represents the number of genes from the input list which are also annotated to the specific pathway. The probability for any given k, where k is an integer in the set of integers from 1 to n, can then be calculated according to the formula:

$$h ( k \vert N ; M ; n ) := P ( X = k ) = \frac{( \begin{matrix}M \\ k\end{matrix} ) ( \begin{matrix}N - M \\ n - k\end{matrix} )}{( \begin{matrix}N \\ n\end{matrix} )} \qquad ( Eq . 1 )$$

In one other specific embodiment, the step of identifying a plurality of stratifying genes based on the stratifying features comprises the steps
performing a statistical analysis,
performing a classification, such as clustering.

In a further embodiment the invention relates to a method, wherein the step of assigning a ranking score to each pathway in the set of pathways comprises the steps of
identifying a number of functional nodes in a pathway, the functional nodes being nodes corresponding to stratifying genes,
identifying a number of hubs in the pathway, the hubs being nodes with a number of connections being larger than an average number of connections per node in the pathway,
identifying a number of important hubs in the pathway, the important hubs being hubs with a number of connections being larger than an average number of connections per node in the pathway,
assigning a ranking score to the pathway, the ranking score being based upon a ratio between the number of functional nodes and a number of nodes and a ratio between the number of important hubs and the number of hubs.

An advantage of such embodiment may be that it can be implemented in a straightforward manner, and that the identification of functional nodes, hubs and important hubs may be simultaneously used for other purposes. In particular, the hubs may be used as indicators, so that a value to be used in a clinical setting, can be calculated by calculating the difference compared to hub values in biomolecular data obtained from a patient sample.

In a particular embodiment, the set of pathways and functional nodes may comprise any one of the pathways and genes given in Table I.

TABLE I

| | | | | | | |
|---|---|---|---|---|---|---|
| | | | Chromosome | | | strand m(-), |
| Gene | ProbeID | Fragment | Num. | Start | End | p(+) |

Resistant vs. Sensitive analysis
akt pathway and wnt signalling pathway

| | | | | | | |
|---|---|---|---|---|---|---|
| GSK3B | 101331.594.482 | MspFrag28471_3_121297088_121297216 | 3 | 121297088 | 121297216 | m |
| FZD1 | 203279.320.922 | MspFrag56995_7_90539644_90539897 | 7 | 90539644 | 90539897 | p |
| CTNNB1 | 92989.616.164 | MspFrag25957_3_41213549_41215233 | 3 | 41213549 | 41215233 | p |

Androgen receptor pathway

| | | | | | | |
|---|---|---|---|---|---|---|
| COX5B | 66677.156.156 | MspFrag18685_2_97721039_97721183 | 2 | 97721039 | 97721183 | p |
| PXN | 340153.701.293 | MspFrag95061_12_119164784_119165946 | 12 | 119164784 | 119165946 | m |
| POU2F1 | 38114.196.208 | MspFrag10698_1_163921158_163921341 | 1 | 163921158 | 163921341 | p |
| CCNE1 | 494022.258.1004 | MspFrag137132_19_34995425_34995795 | 19 | 34995425 | 34995795 | p |
| TMF1 | 98978.256.430 | MspFrag27675_3_69183942_69184077 | 3 | 69183942 | 69184077 | m |
| TMF1 | 98985.584.264 | MspFrag27677_3_69184229_69184352 | 3 | 69184229 | 69184352 | m |
| MAPK1 | 541414.423.597 | MspFrag150099_22_20545088_20545893 | 22 | 20545088 | 20545893 | m |
| PTEN | 280194.192.1014 | MspFrag78241_10_89612949_89613050 | 10 | 89612949 | 89613050 | p |
| NCOA3 | 523517.757.145 | MspFrag145248_20_45563978_45564099 | 20 | 45563978 | 45564099 | p |

gata3 and cytokine gene expression pathway

| | | | | | | |
|---|---|---|---|---|---|---|
| GATA3 | 268815.345.897 | MspFrag74963_10_8137298_8137588 | 10 | 8137298 | 8137588 | p |
| NFATC1 | 468453.516.496 | MspFrag130462_18_75255770_75256048 | 18 | 75255770 | 75256048 | p |
| NFATC1 | 468492.428.622 | MspFrag130473_18_75257144_75257310 | 18 | 75257144 | 75257310 | p |
| NFATC1 | 468505.763.77 | MspFrag130476_18_75257628_75257866 | 18 | 75257628 | 75257866 | p |
| PTX2 | | | | | | |
| CCND2 | 323251.49.807 | MspFrag90182_12_4251165_4251361 | 12 | 4251165 | 4251361 | p |

Normal vs. Tumor analysis
granzyme-mediated apoptosis pathway

| | | | | | | |
|---|---|---|---|---|---|---|
| HMGB2 | 131605.541.163 | MspFrag37005_4_174630489_174630980 | 4 | 174630489 | 174630980 | m |
| SET | 258150.210.34 | MspFrag72158_9_128531004_128531190 | 9 | 128531004 | 128531190 | p |
| APEX1 | 358348.624.922 | MspFrag100127_14_19993268_19993856 | 14 | 19993268 | 19993856 | p |
| DFFB | 5613.251.385 | MspFrag1575_1_3796892_3797034 | 1 | 3796892 | 3797034 | p |

Basic mechanism of action of ppara pparb effects on gene expression

| | | | | | | |
|---|---|---|---|---|---|---|
| PPARD | 169972.513.129 | MspFrag47672_6_35418307_35418428 | 6 | 35418307 | 35418428 | p |
| PPARG | 89713.688.826 | MspFrag24948_3_12304080_12304212 | 3 | 12304080 | 12304212 | p |

IFN a signalling pathway

| | | | | | | |
|---|---|---|---|---|---|---|
| STAT1 | 78675.465.377 | MspFrag22031_2_191704463_191704563 | 2 | 191704463 | 191704563 | m |
| STAT2 | 331937.18.838 | MspFrag92695_12_55040260_55040515 | 12 | 55040260 | 55040515 | m |

Phosphoinosidtides and their downstream targets

| | | | | | | |
|---|---|---|---|---|---|---|
| PRKC2 | | | | | | |
| PLCG1 | 521868.549.775 | MspFrag144778_20_39198472_39198934 | 20 | 39198472 | 39198934 | p |
| GSK3B | 101319.720.720 | MspFrag28469_3_121296382_121296760 | 3 | 121296382 | 121296760 | m |
| PRKCE | 58777.69.739 | MspFrag16462_2_45790699_45790818 | 2 | 45790699 | 45790818 | p |
| GRASP | 329986.258.986 | MspFrag92184_12_50687432_50687714 | 12 | 50687432 | 50687714 | p |

Rho-selective guanine exchange factor akap13 mediates stress fiber formation

| | | | | | | |
|---|---|---|---|---|---|---|
| RHOA | 95515.752.164 | MspFrag26652_3_49424481_49424611 | 3 | 49424481 | 49424611 | m |
| AKAP13 | 389484.262.430 | MspFrag108956_15_83724178_83724944 | 15 | 83724178 | 83724944 | p |

tpo signalling pathway

| | | | | | | |
|---|---|---|---|---|---|---|
| RAF1 | 89805.297.451 | MspFrag24971_3_12680778_12680878 | 3 | 12680778 | 12680878 | m |
| PIOG1 | | | | | | |
| STAT1 | 78676.311.47 | MspFrag22031_2_191704463_191704563 | 2 | 191704463 | 191704563 | m |
| STAT1 | 78675.465.377 | MspFrag22031_2_191704463_191704563 | 2 | 191704463 | 191704563 | m |
| STAT1 | 78674.272.40 | MspFrag22031_2_191704463_191704563 | 2 | 191704463 | 191704563 | m |
| STAT1 | 78664.744.82 | MspFrag22027_2_191703584_191704171 | 2 | 191703584 | 191704171 | m |
| FOS | 367165.85.551 | MspFrag102666_14_74816034_74816285 | 14 | 74816034 | 74816285 | p |

inactivation of gsk3 by akt causes accumulation of b-catenin in alveolar macrophages

| | | | | | | |
|---|---|---|---|---|---|---|
| LEFT | 126420.93.619 | MspFrag35600_4_109446333_109446482 | 4 | 109446333 | 109446482 | m |
| LRP6 | 324917.684.126 | MspFrag90666_12_12311610_12311770 | 12 | 12311610 | 12311770 | m |
| FZD1 | 203266.473.881 | MspFrag56993_7_90539178_90539501 | 7 | 90539178 | 90539501 | p |
| FZD1 | 203271.513.773 | MspFrag56993_7_90539178_90539501 | 7 | 90539178 | 90539501 | p |
| FZD1 | 203292.356.858 | MspFrag56997_7_90540210_90540369 | 7 | 90540210 | 90540369 | p |
| FZD1 | 203272.168.648 | MspFrag56993_7_90539178_90539501 | 7 | 90539178 | 90539501 | p |
| FZD1 | 203270.250.18 | MspFrag56993_7_90539178_90539501 | 7 | 90539178 | 90539501 | p |
| WNT1 | 328477.74.612 | MspFrag91771_12_47659447_47659639 | 12 | 47659447 | 47659639 | p |

wnt signalling

| | | | | | | |
|---|---|---|---|---|---|---|
| GSK3B | 101331.594.482 | MspFrag28471_3_121297088_121297216 | 3 | 121297088 | 121297216 | m |
| PPARD | 169972.513.129 | MspFrag47672_6_35418307_35418428 | 6 | 35418307 | 35418428 | p |
| FZD1 | 203266.473.881 | MspFrag56993_7_90539178_90539501 | 7 | 90539178 | 90539501 | p |
| FZD1 | 203271.513.773 | MspFrag56993_7_90539178_90539501 | 7 | 90539178 | 90539501 | p |
| FZD1 | 203292.356.858 | MspFrag56997_7_90540210_90540369 | 7 | 90540210 | 90540369 | p |
| LRP6 | 324917.684.126 | MspFrag90666_12_12311610_12311770 | 12 | 12311610 | 12311770 | m |
| MAP3K7IPI | | | | | | |

pdgf signalling pathway

| | | | | | | |
|---|---|---|---|---|---|---|
| RAF1 | 89798.41.889 | MspFrag24968_3_12680296_12680502 | 3 | 12680296 | 12680502 | m |
| RAF1 | 89797.225.805 | MspFrag24968_3_12680296_12680502 | 3 | 12680296 | 12680502 | m |
| FOS | 367165.85.551 | MspFrag102666_14_74816034_74816285 | 14 | 74816034 | 74816285 | p |
| FOS | 367163.332.420 | MspFrag102666_14_74816034_74816285 | 14 | 74816034 | 74816285 | p |
| PLCG1 | 521883.758.704 | MspFrag144783_20_39199366_39199498 | 20 | 39199366 | 39199498 | p |
| STAT1 | 78676.311.47 | MspFrag22031_2_191704463_191704563 | 2 | 191704463 | 191704563 | m |
| PDGFRA | 122302.371.217 | MspFrag34299_4_54934710_54935346 | 4 | 54934710 | 54935346 | p |

In a specific embodiment, the ranking may depend on the presence of sub-networks, whereby is to be understood the particular configuration of the functional nodes. In a particular example, it could be that certain sub-networks (i.e. collection of functional nodes) are enriched in certain clinical parameters from a database. Then, pathways containing such enriched sub-networks may be assigned a relatively high ranking score.

In a further embodiment the invention relates to a method for discriminating between normal and tumour samples in cancer diagnostics, wherein the clinical parameter describes a presence of a tumour.

In a further embodiment the invention relates to a method for discriminating between normal and tumour samples in ovarian cancer diagnostics, wherein the clinical parameter describes a presence of a tumour in an ovary. In a further embodiment, the set of pathways includes any one of the pathways in Table II.

TABLE II
Tumor vs. Normal Analysis

| | Entities in | Matched | Matched | |
|---|---|---|---|---|
| Pathway | Pathway | with Chip | with InputList | pValue |
| granzyme a mediated apoptosis pathway | 12 | 9 | 4 | 0.011451 |
| basic mechanism of action of ppara pparb(d) and pparg and effects on gene | 11 | 2 | 2 | 0.011977 |
| expression | | | | |
| rho-selective guanine exchange factor akap13 mediates stress fiber formation | 11 | 2 | 2 | 0.011977 |
| pdgf signaling pathway | 33 | 14 | 5 | 0.013389 |
| effects of calcineurin in keratinocyte differentiation | 18 | 6 | 3 | 0.020292 |
| wnt signaling pathway | 33 | 21 | 6 | 0.021729 |
| phosphoinositides and their downstream targets | 26 | 16 | 5 | 0.024284 |
| visceral fat deposits and the metabolic syndrome | 16 | 3 | 2 | 0.033312 |
| ifn alpha signaling pathway | 12 | 3 | 2 | 0.033312 |
| phospholipase c-epsilon pathway | 26 | 3 | 2 | 0.033312 |
| multi-step regulation of transcription by pitx2 | 35 | 18 | 5 | 0.039652 |
| tpo signaling pathway | 30 | 13 | 4 | 0.045561 |
| inhibition of cellular proliferation by gleevec | 21 | 13 | 4 | 0.045561 |
| nfat and hypertrophy of the heart | 56 | 19 | 5 | 0.049124 |
| inactivation of gsk3 by akt causes accumulation of b-catenin in alveolar | 40 | 19 | 5 | 0.049124 |
| macrophages | | | | |

In a further embodiment the invention relates to a method for predicting responsiveness of a subject with ovarian cancer to a therapy comprising one or more platinum based drugs, wherein the clinical parameter describes a sensitivity towards the therapy comprising one or more platinum based drugs. In a further embodiment, the set of pathways includes any one of the pathways in Table III.

TABLE III
Chemosensitivity Analysis

| | Entities in | Matched | Matched | |
|---|---|---|---|---|
| Pathway | Pathway | with chip | withInputList | pValue |
| AndrogenReceptor | 98 | 72 | 9 | 0.001628 |
| multi-step regulation of transcription by pitx2 | 35 | 18 | 4 | 0.00424 |
| gata3 participate in activating the th2 cytokine genes expression | 16 | 7 | 2 | 0.027084 |
| segmentation clock | 32 | 18 | 3 | 0.029722 |
| PI3K-akt (inactivation of gsk3 by akt causes accumulation of b-catenin | 40 | 19 | 3 | 0.034319 |
| in alveolar macrophages) | | | | |
| Leukocyte transendothelial migration | 36 | 9 | 2 | 0.04414 |
| phosphorylation of mek1 by cdk5/p35 down regulates the map kinase | 17 | 9 | 2 | 0.04414 |
| pathway | | | | |
| Wnt signaling pathway | 33 | 21 | 3 | 0.044541 |

In a further embodiment the invention relates to a method wherein the ranking score is given by a sum of
a ratio between the number of functional nodes and the number of nodes,
a ratio between the number of important hubs and the number of hubs,
a gene set enrichment score,
   wherein the gene set enrichment score is based upon a comparison of the functional nodes and a gene set comprising genes related to the clinical parameter, the gene set enrichment score being indicative of a probability of having the number of functional nodes appearing in a database consisting of clinically relevant genes. The gene set enrichment score can be a p-value based on the Hypergeometric distribution or Fisher's Exact test.

In a further embodiment the invention relates to a method wherein the primary and secondary datasets comprise any one of: a DNA methylation dataset, a gene expression dataset. In a further embodiment, the genes may represent one or more sequences selected from the group consisting of SEQ ID NO: 1-42 (cf. Table IV).

TABLE IV

| | | | Chr | |
|---|---|---|---|---|
| Gene | Probe | Fragment | # | Start |
| DFFB | 5613.251.385 | MspFrag1575_1_3796892_3797034 | 1 | 3796892 |
| POU2F1 | 38114.196.208 | MspFrag10698_1_163921158_163921341 | 1 | 163921158 |
| PRKCE | 58777.69.739 | MspFrag16462_2_45790699_45790818 | 2 | 45790699 |
| COX5B | 66677.156.156 | MspFrag18685_2_97721039_97721183 | 2 | 97721039 |
| STAT1 | 78664.744.82 | MspFrag22027_2_191703584_191704171 | 2 | 191703584 |
| STAT1 | 78675.465.377 | MspFrag22031_2_191704463_191704563 | 2 | 191704463 |
| PPARG | 89713.688.826 | MspFrag24948_3_12304080_12304212 | 3 | 12304080 |
| RAF1 | 89798.41.889 | MspFrag24968_3_12680296_12680502 | 3 | 12680296 |
| RAF1 | 89805.297.451 | MspFrag24971_3_12680778_12680878 | 3 | 12680778 |
| CTNNB1 | 92989.616.164 | MspFrag25957_3_41213549_41215233 | 3 | 41213549 |
| RHOA | 95515.752.164 | MspFrag26652_3_49424481_49424611 | 3 | 49424481 |
| TMF1 | 98978.256.430 | MspFrag27675_3_69183942_69184077 | 3 | 69183942 |
| TMF1 | 98985.584.264 | MspFrag27677_3_69184229_69184352 | 3 | 69184229 |
| GSK3B | 101319.720.720 | MspFrag28469_3_121296382_121296760 | 3 | 121296382 |
| GSK3B | 101331.594.482 | MspFrag28471_3_121297088_121297216 | 3 | 121297088 |
| PDGFRA | 122302.371.217 | MspFrag34299_4_54934710_54935346 | 4 | 54934710 |
| LEF1 | 126420.93.619 | MspFrag35600_4_109446333_109446482 | 4 | 109446333 |
| HMGB2 | 131605.541.163 | MspFrag37005_4_174630489_174630980 | 4 | 174630489 |
| PPARD | 169972.513.129 | MspFrag47672_6_35418307_35418428 | 6 | 35418307 |
| FZD1 | 203266.473.881 | MspFrag56993_7_90539178_90539501 | 7 | 90539178 |
| FZD1 | 203279.320.922 | MspFrag56995_7_90539644_90539897 | 7 | 90539644 |
| FZD1 | 203292.356.858 | MspFrag56997_7_90540210_90540369 | 7 | 90540210 |
| SET | 258150.210.34 | MspFrag72158_9_128531004_128531190 | 9 | 128531004 |
| GATA3 | 268815.345.897 | MspFrag74963_10_8137298_8137588 | 10 | 8137298 |
| PTEN | 280194.192.1014 | MspFrag78241_10_89612949_89613050 | 10 | 89612949 |
| CCND2 | 323251.49.807 | MspFrag90182_12_4251165_4251361 | 12 | 4251165 |
| LRP6 | 324917.684.126 | MspFrag90666_12_12311610_12311770 | 12 | 12311610 |
| WNT1 | 328477.74.612 | MspFrag91771_12_47659447_47659639 | 12 | 47659447 |
| GRASP | 329986.258.986 | MspFrag92184_12_50687432_50687714 | 12 | 50687432 |
| STAT2 | 331937.18.838 | MspFrag92695_12_55040260_55040515 | 12 | 55040260 |
| PXN | 340153.701.293 | MspFrag95061_12_119164784_119165946 | 12 | 119164784 |
| APEX1 | 358348.624.922 | MspFrag_1_00127_14_19993268_19993856 | 14 | 19993268 |
| FOS | 367165.85.551 | MspFrag102666_14_74816034_74816285 | 14 | 74816034 |
| AKAP13 | 389484.262.430 | MspFrag_1_08956_15_83724178_83724944 | 15 | 83724178 |
| NFATC1 | 468453.516.496 | MspFrag130462_18_75255770_75256048 | 18 | 75255770 |
| NFATC1 | 468492.428.622 | MspFrag130473_18_75257144_75257310 | 18 | 75257144 |
| NFATC1 | 468505.763.77 | MspFrag130476_18_75257628_75257866 | 18 | 75257628 |
| CCNE1 | 494022.258.1004 | MspFrag137132_19_34995425_34995795 | 19 | 34995425 |
| PLCG1 | 521868.549.775 | MspFrag144778_20_39198472_39198934 | 20 | 39198472 |
| PLCG1 | 521883.758.704 | MspFrag144783_20_39199366_39199498 | 20 | 39199366 |
| NCOA3 | 523517.757.145 | MspFrag145248_20_45563978_45564099 | 20 | 45563978 |
| MAPK1 | 541414.423.597 | MspFrag150099_22_20545088_20545893 | 22 | 20545088 |

| | | | | | Seq |
|---|---|---|---|---|---|
| | | | | | ID |
| Gene | End | Strand | NCBI_transcriptID | NCBI_proteinID | NO |
| DFFB | 3797034 | p | NM_004402.2 | NP_004393.1 | 1 |
| POU2F1 | 163921341 | p | NM_002697.2 | NP_002688.2 | 2 |
| PRKCE | 45790818 | p | NM_005400.2 | NP_005391.1 | 3 |
| COX5B | 97721183 | p | NM_001862.2 | NP_001853.2 | 4 |
| STAT1 | 191704171 | m | NM_007315.3 | NP_009330.1 | 5 |
| STAT1 | 191704563 | m | | | 6 |
| PPARG | 12304212 | p | NM_005037.5 | NP_005028.4 | 7 |
| RAF1 | 12680502 | m | NM_002880.3 | NP_002871.1 | 8 |
| RAF1 | 12680878 | m | | | 9 |
| CTNNB1 | 41215233 | p | NM_007614.3 | NP_031640.1 | 10 |
| RHOA | 49424611 | m | NM_001664.2 | NP_001655.1 | 11 |
| TMF1 | 69184077 | m | NM_007114.2 | NP_009045.2 | 12 |
| TMF1 | 69184352 | m | | | 13 |
| GSK3B | 121296760 | m | NM_002093.3 | NP_002084.2 | 14 |
| GSK3B | 121297216 | m | | | 15 |
| PDGFRA | 54935346 | p | NM_006206.4 | NP_006197.1 | 16 |
| LEF1 | 109446482 | m | NM_016269.4 | NP_057353.1 | 17 |
| HMGB2 | 174630980 | m | NM_002129.3 | NP_002120.1 | 18 |
| PPARD | 35418428 | p | NM_006238.4 | NP_006229.1 | 19 |
| FZD1 | 90539501 | p | NM_003505.1 | NP_003496.1 | 20 |
| FZD1 | 90539897 | p | | | 21 |
| FZD1 | 90540369 | p | | | 22 |
| SET | 128531190 | p | NM_001122821.1 | NP_001116293.1 | 23 |
| GATA3 | 8137588 | p | NM_001002295.1 | NP_001002295.1 | 24 |
| PTEN | 89613050 | p | NM_000314.4 | NP_000305.3 | 25 |
| CCND2 | 4251361 | p | NM_001759.3 | NP_001750.1 | 26 |
| LRP6 | 12311770 | m | NM_002336.2 | NP_002327.2 | 27 |
| WNT1 | 47659639 | p | NM_005430.3 | NP_005421.1 | 28 |
| GRASP | 50687714 | p | NM_138894.1 | NP_620249.1 | 29 |
| STAT2 | 55040515 | m | NM_005419.3 | NP_005410.1 | 30 |
| PXN | 119165946 | m | NM_001080855.1 | NP_001074324.1 | 31 |
| APEX1 | 19993856 | p | NM_001641.2 | NP_001632.2 | 32 |
| FOS | 74816285 | p | NM_005252.3 | NP_005243.1 | 33 |
| AKAP13 | 83724944 | p | NM_006738.4 | NP_006729.4 | 34 |
| NFATC1 | 75256048 | p | NM_006162.3 | NP_006153.2 | 35 |
| NFATC1 | 75257310 | p | | | 36 |
| NFATC1 | 75257866 | p | | | 37 |
| CCNE1 | 34995795 | p | NM_001238.1 | NP_001229.1 | 38 |
| PLCG1 | 39198934 | p | NM_002660.21 | NP_002651.2 | 39 |
| PLCG1 | 39199498 | p | | | 40 |
| NCOA3 | 45564099 | p | NM_181659.2 | NP_858045.1 | 41 |
| MAPK1 | 20545893 | m | NM_002745.4 | NP_002736.3 | 42 |

In a further embodiment the invention relates to a method wherein the primary and secondary datasets comprise methylation data and wherein the functional nodes represent genes which are hypermethylated and/or genes which are hypomethylated.

In a further embodiment the invention relates to an assay according to the second aspect of the invention for analysing an expression pattern of said genes, such as room temperature polymerase chain reaction (RT-PCR), RNA sequencing, gene expression microarrays.

In a further embodiment the invention relates to an assay according to the second aspect of the invention for analysing a methylation pattern of said target nucleic acids, such as by using methylation specific PCR (MSP), bisulfite sequencing, microarrays, direct sequencing, such as implemented by Pacific Biosciences(R).

To sum up, a method for assigning ranking scores to pathways in a set of pathways for classifying patients is disclosed. The method comprises the steps of comparing biomolecular datasets from different groups of patients and performing an analysis in order to assign ranking scores to pathways in a set of pathways. Furthermore, a method for using cancer pathway evaluation to support clinical decision making is disclosed. This assessment is further used for stratifying ovarian cancer patients based on chemosensitivity to platinum based drugs, the standard chemotherapy. We present the method for evaluation and ranking of the most relevant pathways responsible for platinum sensitivity. Clinical decision support software system should be able to then visualize this information for a clinician, contextualize it within a patient data set and help make a final decision on the potential responsiveness.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

### Example 1

Interrogating chemosensitivity in ovarian cancer patients using pathway analysis.

### Description of the Data

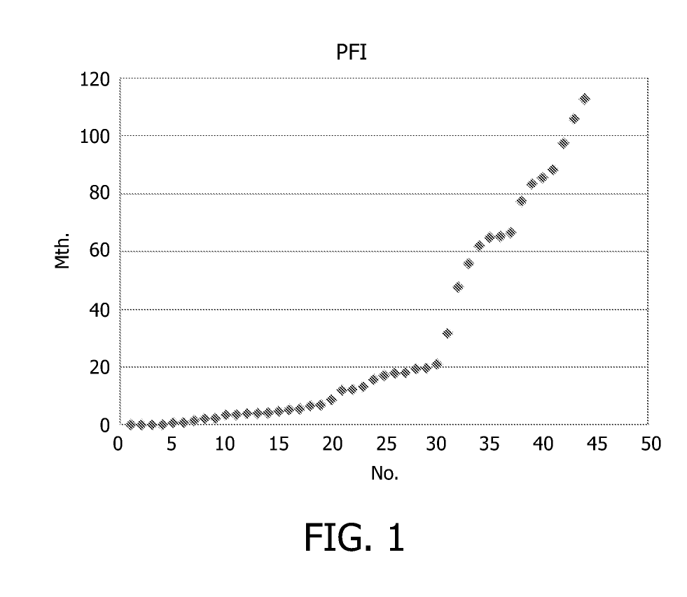

Our goal was to find differentially regulated pathways based on methylation information from CpG island loci on a genome wide scale to study platinum sensitivity in ovarian cancer samples. We have processed 44 ovarian cancer samples, all grade III, histologically classified as serous carcinoma. The platinum free interval in our sample set varies from 0 to 112 months (see FIG. 1). The traditional definition for the platinum free interval categorizes patients with PFI less than 6 months as platinum-resistant and more than 6 months as platinum-sensitive. We performed a statistical analysis of the resistant vs. sensitive on the geometric mean of CpG island microarray data, which originates from a Methylation Oligonucleotide Microarray Analysis (MOMA). The inventors of the present invention have participated in the development of a CpG island microarray called MOMA: Methylation Oligonucleotide Microarray Analysis for the use of finding differentially methylated patterns in breast and ovarian cancer. The array, Methylation Oligonucleotide Microarray Analysis (MOMA) interrogates about 150,000 loci on 270,000 known CpG islands, across the whole genome for differential methylation.

In the context of the present invention, the term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 kb in length.

FIG. 1 shows clinical data: Platinum Free Interval (PFI) for all samples.

### Statistical Data Analysis

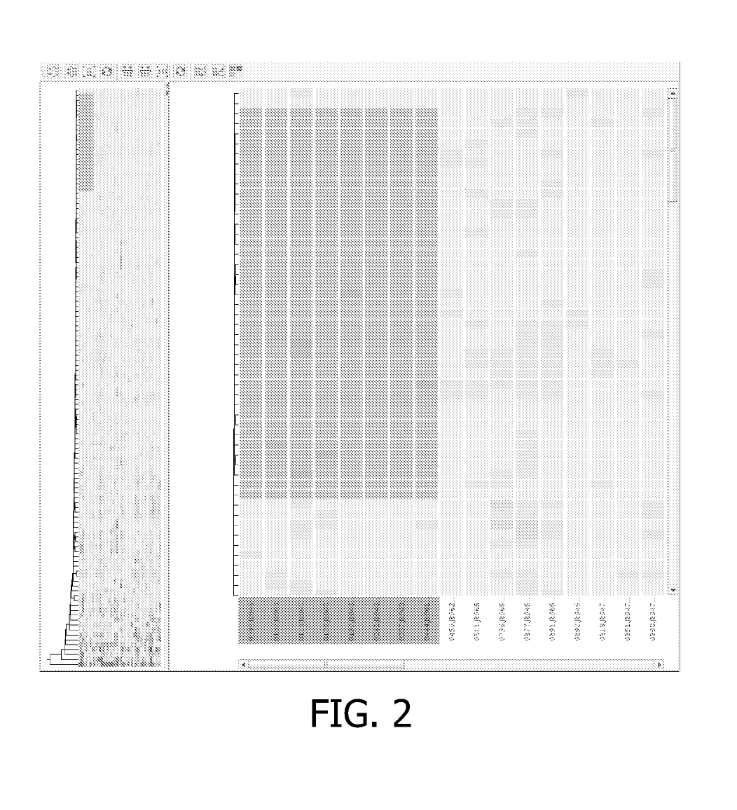

Before applying pathway analysis, we start with a standard unpaired t-test, followed by clustering. We experimented with different levels of differential methylation change, and obtained a signature containing 5703 differentially methylated loci at p-value 0.05 and a fold change of 1.1. FIG. 2 shows the clustering dendrogram using the aggregate geometric mean signal value for the statistically significant differentially methylated loci between the resistant and sensitive groups.

FIG. 2 shows hierarchical clustering of all loci after a t-test (p-val 0.05) and fold change>1.1.

Although these are statistically significant loci, the subsequent hierarchical clustering on all the patients revealed a pattern that seemed to result in clusters that did not have big inter-cluster difference. Indeed, we observed that in our data set there is a continuum of PFI from 6 months onward up to 112 months, and we cannot expect that these patients (the ones between 6 and 30 months) to have a completely distinct molecular profile from the patients whose PFI is less than 6 months. Hierarchical clustering, on the differentially methylated loci obtained from a t-test where fold change is greater than 1.1 and p-value is 0.05 is shown in FIG. 2.

### Pathway Analysis

Based on this initial set that describes the difference between resistance and sensitivity to platinum based drugs in ovarian cancer we performed pathway analysis using a commercially available tool in GeneSpring GX 10.0. The FindSignificantPathways tool was used to identify pathways that are critical in distinguishing between the early-resistant and sensitive samples based on the filtered fragment-list.

FindSignificantPathway takes an entity list (could be methylation probe IDs, or Affymetrix gene expression probes or identifiers that can be linked to an Entrez gene ID or gene symbol) as an input and finds all pathways from a collection which have significant overlap with that entity list. Here, overlap denotes the number of common entities between the list and the pathway. Commonness is determined via the presence of a shared identifier, i.e., Entrez Gene ID, or gene symbol. Once the number of common entities is determined, the p-value computation is based on the Hypergeometric method or the Fisher's exact test. The results are output as a table which shows the names of the pathways, the total number of nodes in the pathway, the number of genes from the input list that belong to the pathway and the p-value. The p-value shows the probability of getting that particular pathway by chance when this set of entity list is used.

Pathways showing significant overlap with genes (entities) in the gene list (entity list) selected for analysis are displayed in Table III.

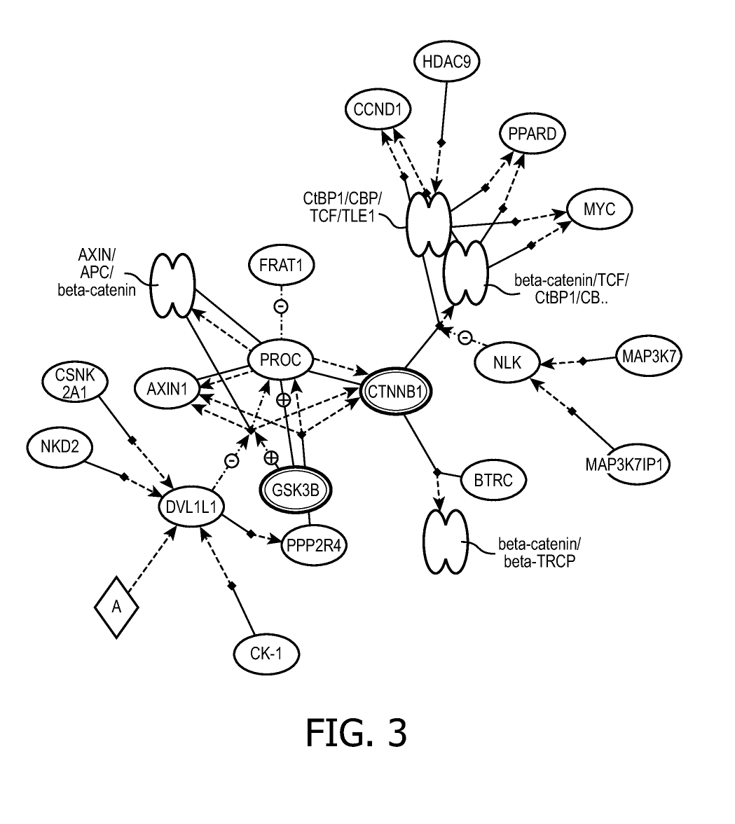

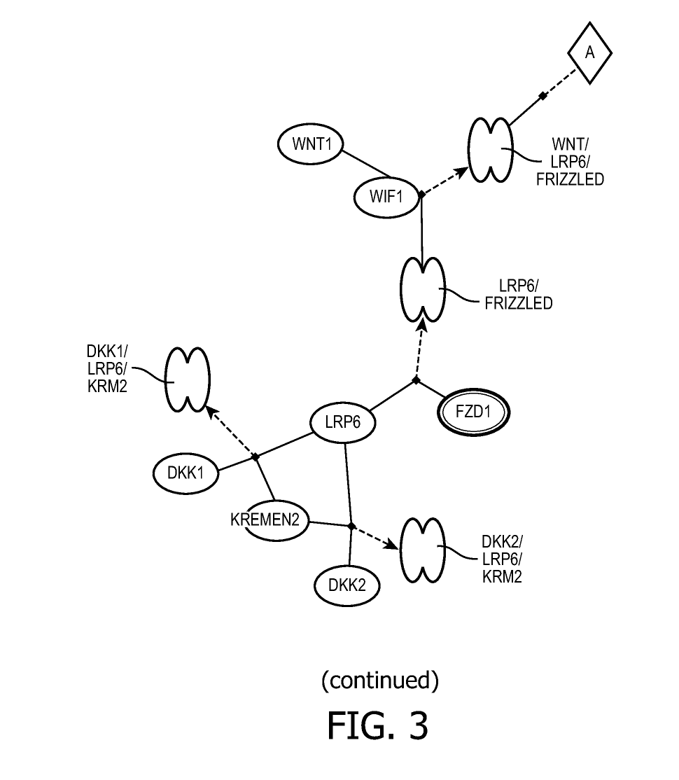

FIG. 3 shows Wnt Pathway and functional nodes. The genes with blue halo are the 3 genes from the input list (cf. Table III) that belong to Wnt pathway. In FIGS. 3-5, the elongated elliptical entities represent proteins, the smaller circular entities represent small molecules and the larger entities which appear composed of two vertically elongated ellipsoids represent complexes.

### Example 2

Interrogating tumor vs. normal samples using pathway analysis.

### Description of the Data

We performed statistical analysis of normal vs. tumors on the geometric mean of MOMA data. We performed unpaired t-test, wilcoxon-rank sum test and a linear Bayesian model-based analysis with leave one out validation to identify differentially methylated probes. Similar pathway analysis as applied to resistant vs. sensitive patients (cf. Example 1) was applied to the differentially methylated probes.

Table II shows significant pathways distinguishing tumor vs. normal samples,

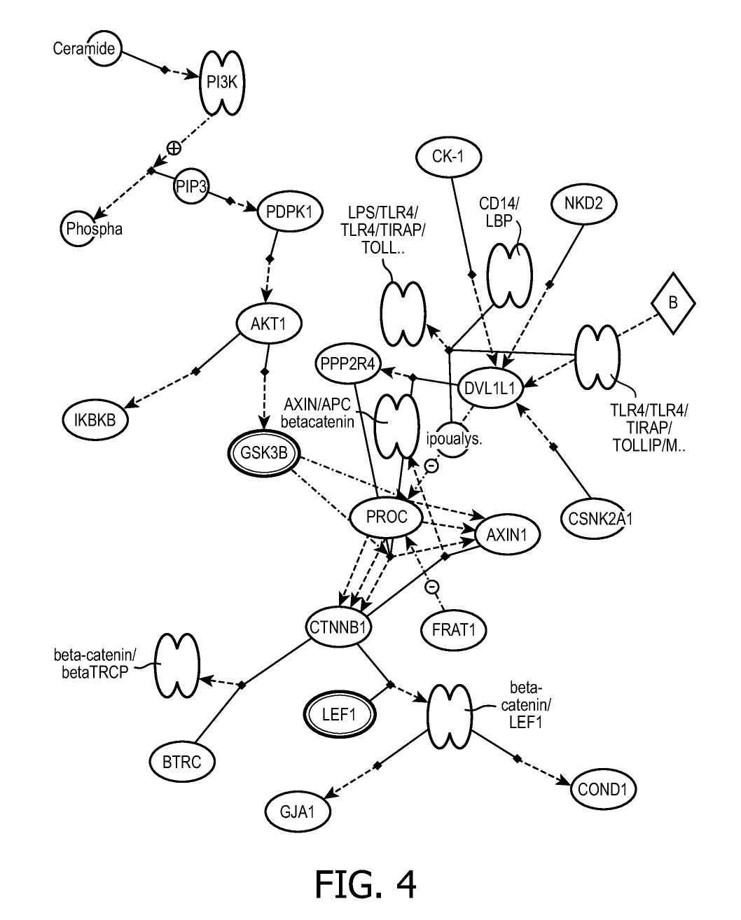

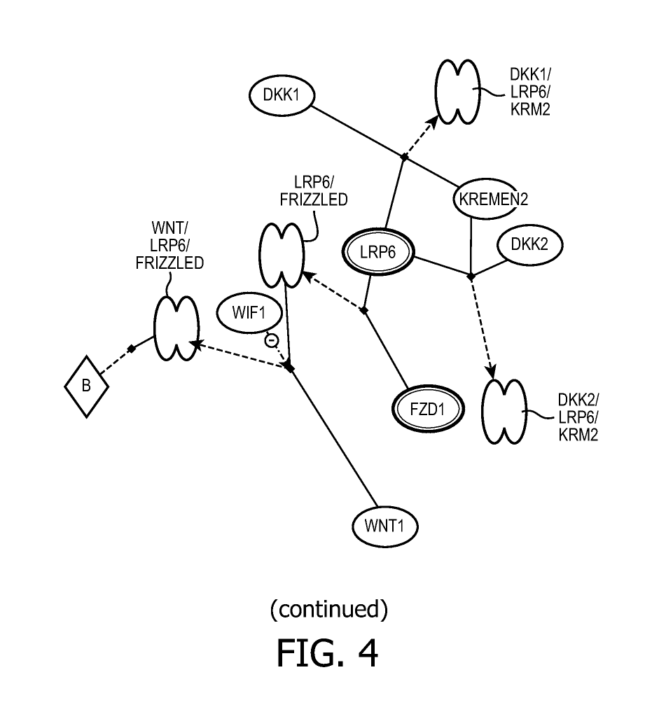

FIG. 4 shows inactivation of gsk3 by akt causes accumulation of b-catenin in alveolar macrophage,

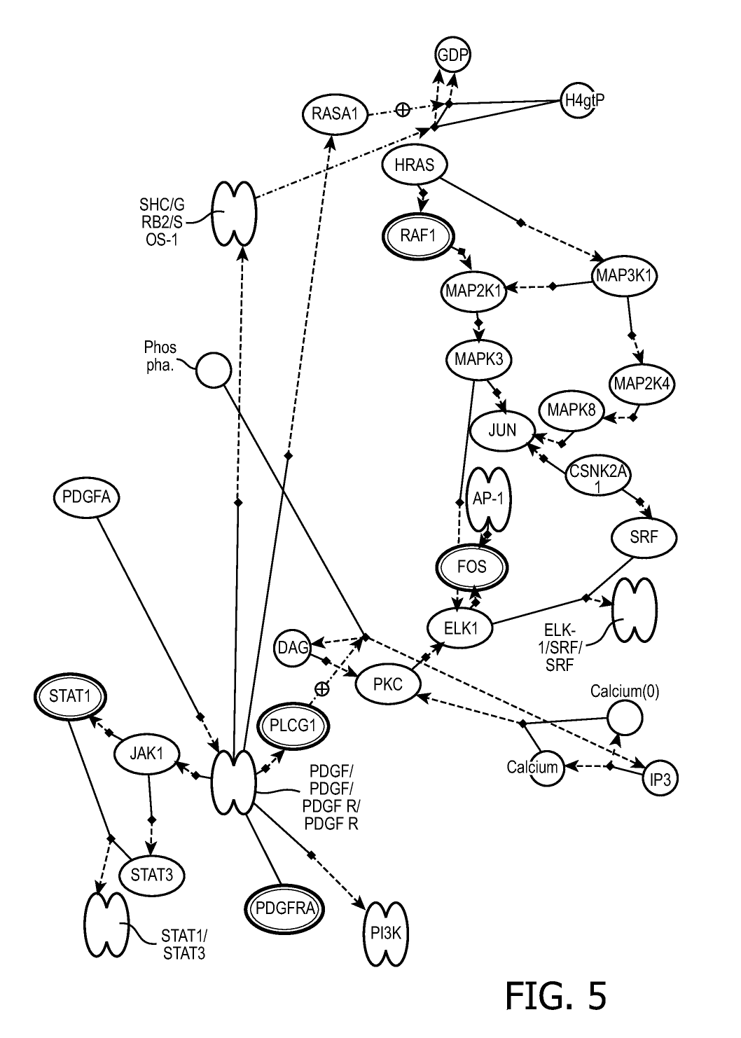

FIG. 5 shows the PDGF signalling pathway deemed significant in tumor vs. normal analysis.

Description of a method according to an embodiment of the invention

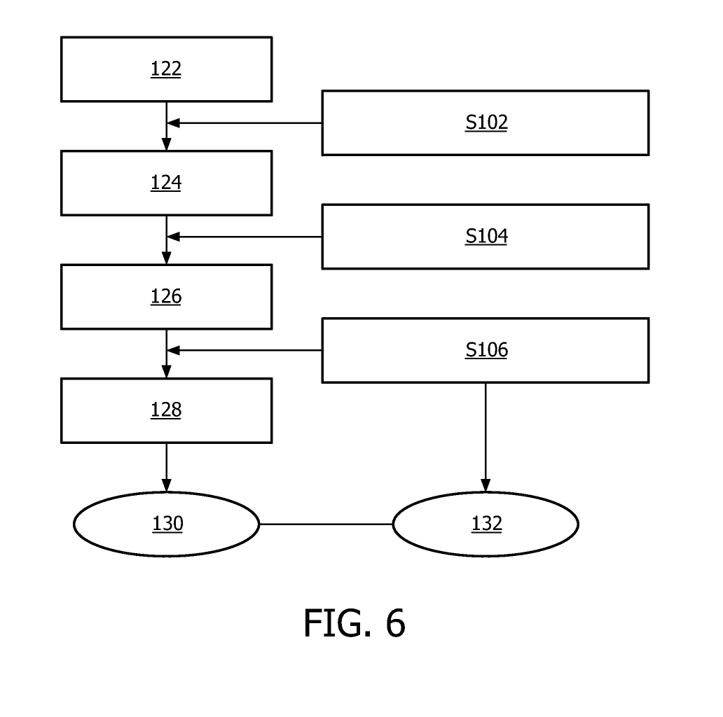

FIG. 6 shows a flow chart according to an embodiment according of the invention where primary and secondary datasets 122 are given, which primary and secondary datasets may be high throughput data, representing gene expression or methylation data. Statistical techniques are applied in a method step S102 in order to identify stratifying features 124 in the primary and secondary datasets 122. As a result, a list of stratifying features 124 is obtained, which list may be a list of stratifying genes. Ranking scores are assigned to each pathway in a plurality of pathways in a subsequent step S104, which step results in a set of ranked pathways 126, said ranking being based upon the plurality of stratifying genes. For a pathway in the set of ranked pathways, functional nodes are identified in yet another step S106, which functional nodes may, for example, be statistically significant hyper-methylated or hypo-methylated nodes. This may form the basis of a visualization 128 of the specific pathway, showing the functional nodes in the pathway. Furthermore, the identification of the functional nodes may serve as input to an assay 132 for analysing one or a combination of the genes taken from the group consisting of stratifying genes which are also present in the pathway, i.e., functional nodes. The visualization 128 may itself serve as input to a clinical decision support system 130.

SEQUENCE LISTING

| | DNA/AMINO | |
|---|---|---|
| SEQ ID NO | ACID (AA) | NAME |
| SEQ ID NO 1 | DNA | DFFB_5613.251.385 |
| SEQ ID NO 2 | DNA | POU2F1_38114.196.208 |
| SEQ ID NO 3 | DNA | PRKCE_58777.69.739 |
| SEQ ID NO 4 | DNA | COX5B_66677.156.156 |
| SEQ ID NO 5 | DNA | STAT1_78664.744.82 |
| SEQ ID NO 6 | DNA | STAT1_78675.465.377 |
| SEQ ID NO 7 | DNA | PPARG_89713.688.826 |
| SEQ ID NO 8 | DNA | RAF1_89798.41.889 |
| SEQ ID NO 9 | DNA | RAF1_89805.297.451 |
| SEQ ID NO 10 | DNA | CTNNB1_92989.616.164 |
| SEQ ID NO 11 | DNA | RHOA_95515.752.164 |
| SEQ ID NO 12 | DNA | TMF1_98978.256.430 |
| SEQ ID NO 13 | DNA | TMF1_98985.584.264 |
| SEQ ID NO 14 | DNA | GSK3B_101319.720.720 |
| SEQ ID NO 15 | DNA | GSK3B_101331.594.482 |
| SEQ ID NO 16 | DNA | PDGFRA_122302.371.217 |
| SEQ ID NO 17 | DNA | LEF1_126420.93.619 |
| SEQ ID NO 18 | DNA | HMGB2_131605.541.163 |
| SEQ ID NO 19 | DNA | PPARD_169972.513.129 |
| SEQ ID NO 20 | DNA | FZD1_203266.473.881 |
| SEQ ID NO 21 | DNA | FZD1_203279.320.922 |
| SEQ ID NO 22 | DNA | FZD1_203292.356.858 |
| SEQ ID NO 23 | DNA | SET_258150.210.34 |
| SEQ ID NO 24 | DNA | GATA3_268815.345.897 |
| SEQ ID NO 25 | DNA | PTEN_280194.192.1014 |
| SEQ ID NO 26 | DNA | CCND2_323251.49.807 |
| SEQ ID NO 27 | DNA | LRP6_324917.684.126 |
| SEQ ID NO 28 | DNA | WNT1_328477.74.612 |
| SEQ ID NO 29 | DNA | GRASP_329986.258.986 |
| SEQ ID NO 30 | DNA | STAT2_331937.18.838 |
| SEQ ID NO 31 | DNA | PXN_340153.701.293 |
| SEQ ID NO 32 | DNA | APEX1_358348.624.922 |
| SEQ ID NO 33 | DNA | FOS_367165.85.551 |
| SEQ ID NO 34 | DNA | AKAP13_389484.262.430 |
| SEQ ID NO 35 | DNA | NFATC1_468453.516.496 |
| SEQ ID NO 36 | DNA | NFATC1_468492.428.622 |
| SEQ ID NO 37 | DNA | NFATC1_468505.763.77 |
| SEQ ID NO 38 | DNA | CCNE1_494022.258.1004 |
| SEQ ID NO 39 | DNA | PLCG1_521868.549.775 |
| SEQ ID NO 40 | DNA | PLCG1_521883.758.704 |
| SEQ ID NO 41 | DNA | NCOA3_523517.757.145 |
| SEQ ID NO 42 | DNA | MAPK1_541414.423.597 |

1. A method for assigning ranking scores to pathways in a set of pathways for classifying subjects, said method comprising the steps of
distinguishing a plurality of primary subjects from a corresponding plurality of secondary subjects by means of a clinical parameter relevant to cancer, which differs between the primary and the secondary subjects,
obtaining a plurality of primary datasets comprising biomolecular features from the plurality of primary subjects,
obtaining a plurality of secondary datasets comprising biomolecular features from the plurality of secondary subjects,
identifying a plurality of stratifying features (124) in the primary and secondary datasets, wherein the stratifying features (124) are biomolecular features which differ in a statistically significant manner between the primary and secondary datasets (S102),
identifying a plurality of stratifying genes corresponding to the stratifying features,
assigning a ranking score to each pathway in the set of pathways (S104) thereby providing a set of ranked pathways (126), said ranking being based upon the plurality of stratifying genes,
wherein the step of assigning a ranking score to each pathway in the set of pathways comprises the steps of
identifying a number of functional nodes in a pathway, the functional nodes being nodes corresponding to stratifying genes,
identifying a number of hubs in the pathway, the hubs being nodes with a number of connections being larger than an average number of connections per node in the pathway,
identifying a number of important hubs in the pathway, the important hubs being hubs with a number of connections being larger than an average number of connections per hub in the pathway,
assigning a ranking score to the pathway, the ranking score being based upon a ratio between the number of functional nodes and a number of nodes and a ratio between the number of important hubs and the number of hubs.

2. A method for assigning ranking scores to pathways in a set of pathways according to claim 1, wherein the step of assigning a ranking score to each pathway in the set of pathways comprises calculating a significance value for each pathway, said significance value being based upon a number of common genes between the plurality of stratifying genes and the pathway.

3. (canceled)

4. A method for assigning ranking scores to pathways in a set of pathways according to claim 1 for discriminating between normal and tumour samples in cancer diagnostics, wherein the clinical parameter describes a presence of a tumour.

5. A method for assigning ranking scores to pathways in a set of pathways according to claim 1 for discriminating between normal and tumour samples in ovarian cancer diagnostics, wherein the clinical parameter describes a presence of a tumour in an ovary.

6. A method for assigning ranking scores to pathways in a set of pathways according to claim 1 for predicting responsiveness of a subject with ovarian cancer to a therapy comprising one or more platinum based drugs, wherein the clinical parameter describes a sensitivity towards the therapy comprising one or more platinum based drugs.

7. A method for assigning ranking scores to pathways in a set of pathways according to claim 1, wherein the ranking score for a pathway is given by a sum of
a ratio between the number of functional nodes and the number of nodes in the pathway,
a ratio between the number of important hubs and the number of hubs in the pathway,
a gene set enrichment score,
wherein the gene set enrichment score is based upon a comparison of the functional nodes in the pathway and a gene set comprising genes related to the clinical parameter, the gene set enrichment, score being indicative of a probability of having the number of functional nodes appearing in a database consisting of clinically relevant genes.

8. A method for assigning ranking scores to pathways in a set of pathways according to claim 1, wherein the primary and secondary datasets comprise any one of: a DNA methylation dataset, a gene expression dataset.

9. A method for assigning ranking scores to pathways in a set of pathways according to claim 1, wherein the primary and secondary datasets comprise methylation data and wherein the functional nodes represent genes which are hypermethylated and/or genes which are hypomethylated.

10-12. (canceled)

13. A method for classifying a subject, said method comprising
obtaining a subject dataset comprising biomolecular data of a target nucleic acid comprising one or a combination of the genes taken from a group consisting of stratifying genes according to claim 1 and their regulatory regions,
identifying the pathway according to claim 1, which is assigned the highest ranking score,
accessing a database comprising database values of the stratifying features corresponding to hubs of the pathway, which is assigned the highest ranking score, which is identified according to claim 1,
calculating a subject classification score based on the difference between database values of the stratifying features corresponding to the hubs and values of corresponding features in the subject dataset.

14. A clinical decision support system comprising
an input for providing a subject dataset comprising biomolecular data of a target nucleic acid comprising one or a combination of the genes taken from a group consisting of stratifying genes according to claim 1 and their regulatory regions,
a computer program product for enabling a processor to carry out the method of claim 15,
an output for outputting the subject classification score.

15. A computer program product for enabling a processor to carry out the method of claim 13.
